(19) United States
(12) Patent Application Publication
(10) Pub. No.: US 2015/0112307 A1
(43) Pub. Date: Apr. 23, 2015

(54) DUAL RAPID EXCHANGE CATHETERS, SYSTEMS, AND METHODS

(71) Applicant: Wayne Margolis Family Partnership, Ltd., Nederland, TX (US)

(72) Inventor: Wayne Margolis, Nederland, TX (US)

(73) Assignee: Wayne Margolis Family Partnership, Ltd., Nederland, TX (US)

(21) Appl. No.: 14/135,879

(22) Filed: Dec. 20, 2013

Related U.S. Application Data
(63) Continuation of application No. 14/057,983, filed on Oct. 18, 2013.

(51) Int. Cl. A61M 25/00; A61F 2/954; A61M 25/01

(52) U.S. Cl. CPC A61M 25/0029; A61M 25/0169; A61M 25/0108; A61M 25/0102; A61F 2/954
USPC 604/510; 604/523; 604/529; 604/508; 604/170.02

(57) ABSTRACT

In one instance, a catheter for replacing or adding a guide wire into a patient is provided. The catheter includes a first rapid exchange lumen, a second rapid exchange lumen, and a releasable wire-securing device. A second guide wire is placed in the second rapid exchange lumen and secured into position. The catheter is then advanced along the first guide wire until in position, and then the second guide wire is released from the wire-securing device and advanced into the desired position. The catheter is then removed. Other catheters and methods are also disclosed.

### CROSS REFERENCE TO RELATED APPLICATIONS

This application is a Continuation of U.S. patent application Ser. No. 14/057,983 filed on Oct. 18, 2013, entitled “DUAL RAPID EXCHANGE CATHETERS, SYSTEMS, AND METHODS,” which is incorporated herein by reference for all purposes.

### TECHNICAL FIELD

This application is directed, in general, to intravascular catheters, and more specifically, but not by way of limitation, to dual rapid exchange catheters, systems, and methods.

### BACKGROUND

Some patients develop issues with restricted or blocked flow in parts of their vascular system that must be addressed. A common example is restriction or blockage of one or more coronary arteries. In the past, this situation was address almost exclusively by coronary artery bypass surgery (CABG). Beginning in the late 1970s, balloon angioplasty of the coronary artery, or percutaneous transluminal coronary angioplasty (PTCA) began to be used. PTCA is now referred to as percutaneous coronary intervention (PCI). Almost simultaneously similar techniques were developed to treat restriction or blockages of other blood vessels now referred to as percutaneous vascular interventions (PVI) and non-vascular restriction or blockages such as those involving but not limited to the biliary system and tracheal/bronchial system. What all these various procedures usually have in common is the initial placement of a guide wire across the restriction or blockages. The guide wire is then used as a rail to deliver the interventional device.

Percutaneous interventions (PCI) may involve using balloon catheters, stents, and atherectomy devices. In the typical PCI situation today, a small balloon catheter is inserted into an artery in the leg or arm and directed to the narrowing of the artery. In the typical percutaneous interventional situation today, a guide sheath or guide catheter is inserted percutaneously into an artery, vein, or other in situ structure that will allow access to the target lesion. A guide wire is then used to cross the restriction or blockage. A balloon catheter or other interventional device is then inserted over the guide wire and directed across the narrowing of the artery, vein or other structure. There in the case of the balloon, the balloon is inflated to enlarge the narrowed portion of the artery.

In addition to angioplasty, beginning in the 1990s, many patients have been treated with stents. Stents are typically wire mesh tubing that is delivered with a percutaneous intervention balloon. The stent is over the balloon. As the balloon inflates it moves the stent out to help provide a scaffold for the artery and to maintain blood flow through the artery. The stents may be medicated, or drug-eluting stents, that help reduce the occurrence of blockage, or restenosis.

While great advances have been made in these interventional procedures and others, improvement is still desirable.

### DESCRIPTION OF THE DRAWINGS

Illustrative embodiments of the present invention are described in detail below with reference to the attached drawing figures, which are incorporated by reference herein and wherein:

FIG. 1 is a schematic perspective view of an illustrative embodiment of a catheter for introducing a second guide wire into a patient;

FIG. 2 is a schematic perspective view of an illustrative embodiment of a catheter for introducing a second guide wire into a patient with a few small changes from that of FIG. 1;

FIG. 3 is a schematic cross section taken along line 3-3 in FIG. 1;

FIG. 4 is a schematic cross section taken along line 4-4 in FIG. 1;

FIG. 5 is a schematic cross section taken along line 5-5 in FIG. 1;

FIG. 6 is a schematic perspective view of a portion of an illustrative embodiment of a catheter for introducing a second guide wire into a patient showing each exit axis;

FIG. 7 is a schematic perspective view of a portion of an illustrative embodiment of a catheter for introducing a second guide wire into a patient showing an exit axis for the second port of the second rapid exchange lumen at an angle to the longitudinal axis of the first port of the first rapid exchange lumen or the longitudinal catheter body;

FIG. 8 is a schematic cross section taken along line 8-8 in FIG. 1 for another illustrative embodiment;

FIG. 9 is a schematic cross section taken along line 9-9 in FIG. 1 for another illustrative embodiment;

FIG. 10 is a schematic cross section taken along a second portion of the catheter of FIG. 9; and

FIG. 11 is a schematic elevation view of a proximal portion of the catheter of FIGS. 9-10 showing a variable stiffening member.

### DETAILED DESCRIPTION

In the following detailed description of the preferred embodiments, reference is made to the accompanying drawings that form a part hereof, and in which is shown, by way of illustration, specific embodiments in which the invention may be practiced. These embodiments are described in sufficient detail to enable those skilled in the art to practice the invention, and it is understood that other embodiments may be utilized and that logical structural, mechanical, electrical, and chemical changes may be made without departing from the spirit or scope of the invention. To avoid detail not necessary to enable those skilled in the art to practice the invention, the description may omit certain information known to those skilled in the art. The following detailed description is, therefore, not to be taken in a limiting sense, and the scope of the present invention is defined only by the claims.

Unless otherwise indicated, as used throughout this document, “or” does not require mutual exclusivity.

In carrying out various intravascular procedures, there are occasions when it is desirable to add a second guide wire after a first guide wire has been positioned in a vessel lumen. This may be because a stiffer wire is needed to accommodate a balloon catheter through a blockage or to access a side branch/bifurcation, or some other reasons. The disclosure provides a catheter and methods for introducing a second guide wire using the first guide wire and doing so relatively quickly and with the avoidance of entanglement of the two guide wires.

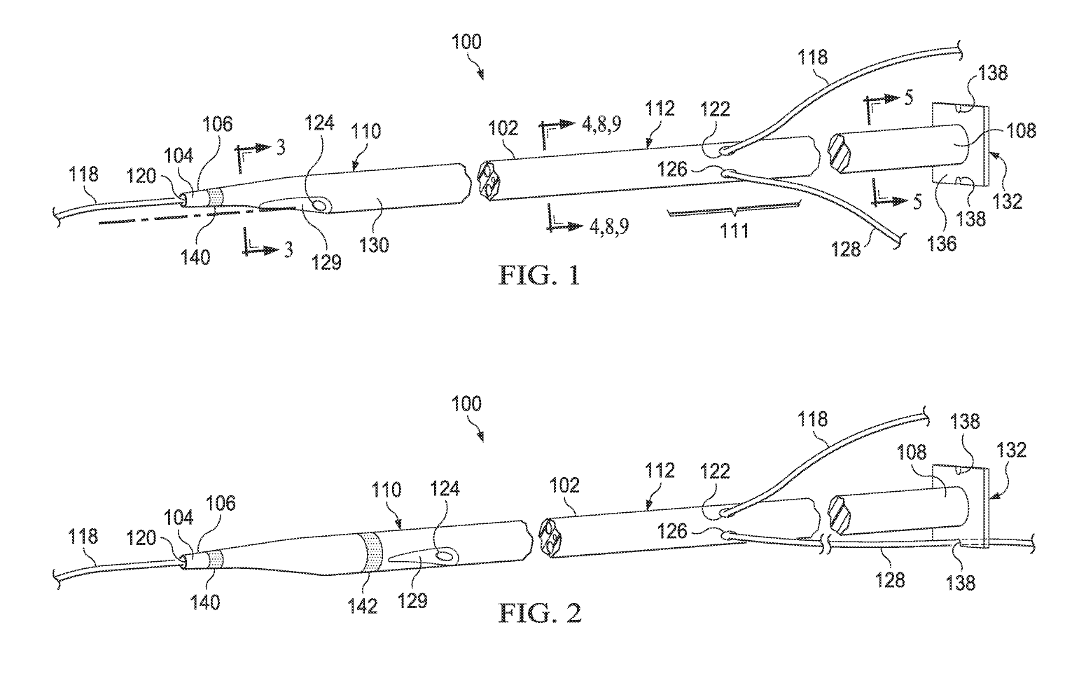

Referring now to the drawings, and initially to FIG. 1, a catheter 100 for introducing a second guide wire into a patient is presented. The catheter 100 includes a longitudinal catheter body 102 having a distal end 104 with a catheter tip 106 that leads on introduction into the patient. The distal end 104 may be tapered for ease of introduction and movement within a vessel lumen. The longitudinal catheter body 102 also has a proximal end 108. The longitudinal catheter body 102 has a first portion 110 and a second portion 112 that is proximal to the first portion. A medial portion 111 is located between the distal end 104 and the proximal end 108. In one embodiment, the medial portion 111 is closer to the distal end 104 than the proximal end.

The longitudinal catheter body 102 may be formed from any material that is flexible enough to navigate the tortuous path of a vessel lumen and yet stiff enough to contain the one or more guide wires. For example, the longitudinal catheter body 102 may be formed from polyethylene, polyamide, polyamide, polyvinylchloride (PVC), polyester and other high-strength polymers, radiation cross-linked polyethylene, nylons, polyethylene terephthalate (PET), polytetrafluoroethylene (PTFE), and polyether block amide (PEBAX), etc. The catheter body 102 may also be formed from braided material (wire, stainless steel, nylon ribbon, fiber braiding, etc.). Other similar materials may also be used.

The longitudinal catheter body 102 is formed with a first rapid exchange lumen 114 and a second rapid exchange lumen 116 as shown clearly in the cross section of FIG. 4. The two rapid exchange lumens 114 and 116 may be configured as shown in FIG. 4 or be in closer proximity to each other in a figure-eight type pattern. The thickness of the longitudinal catheter body 102 can vary based on the catheter configuration and the materials used in construction. Each rapid exchange lumen 114, 116 starts at the distal end 104 but finishes before the proximal end 108 of the catheter 100. This arrangement allows for rapid exchange of the catheter 100 over the same guide wire. The first rapid exchange lumen 114 is formed in the first portion 110 of the longitudinal catheter body 102. The first rapid exchange lumen 114 has a first port 120 proximate the catheter tip 106 and a second port 122 on the medial portion 111 of the longitudinal catheter body 102.

The second rapid exchange lumen 116 is formed in the first portion 110 of the longitudinal catheter body 102. The second rapid exchange lumen 116 has a first port 124 proximate the first end or distal end 104 of the longitudinal catheter body 102 that is proximal to the first port 120 of the first rapid exchange lumen 114. The second rapid exchange lumen 116 has a second port 126 on the medial portion 111 of the longitudinal catheter body 102. A second guide wire 128 may be positioned at least partially within the second rapid exchange lumen 116 during introduction into the patient. The lengths of the rapid exchange lumens 114, 116 may vary according to the application, but generally would be the same length as commercially available single rapid exchange catheters available today.

A first guide wire 118 comes out relatively (compared to over-the-wire arrangement) closer to the distal end 104 of the catheter 100. The second wire also comes out relatively closer to the distal end. For example, the wires may come out on a proximal end through second ports 122, 126 that each are located in general but not limited to 5 to 70% of the length of the catheter going from the tip 106. The exits or ports may be at the same location or may be staggered.

The first port 124 of the second rapid exchange lumen 116 may be fairly close to the first port 120 of the first rapid exchange lumen 114 as suggested in FIG. 1 or even closer. The first port 124 of the second rapid exchange lumen 116 may be relatively further away from the first port 120 of the first rapid exchange lumen 114 as suggested in FIG. 2. For example, without limitation, the first port 124 of the second rapid exchange lumen 116 may be displaced proximally from the first port 120 of the first rapid exchange lumen 114 by percentage of the length (longitudinal dimension) of the longitudinal catheter body 102. The percentage may be anything from 0-50% or more inclusive of any numbers there between, e.g., 5%, 10%, 15%, 20%, etc.

The first port 124 of the second rapid exchange lumen 116 may be formed in many ways. Preferably the first port 124 has a low profile to make introduction of the catheter 100 into a vessel lumen as easy as possible. The first port 124 may be indented or have an angled entry way 129 compared to other portions of an exterior 130 of the catheter 100. The first port 124 may have an oval, round, or angular shape as the port's entry way is cut or formed in the longitudinal catheter body 102.

The exterior 130 is typically a smooth surface to facilitate movement of the catheter 100 within vessel lumens. Also, the exterior 130 of the catheter 100 may coated with a lubricious coating to further facilitate movement of the catheter. Similarly, the first rapid exchange lumen 114 and the second rapid exchange lumen 116 may have a lubricious coating applied. Possible coatings include hydrophilic coatings, aqueous-based lubricious coatings, or any other coating used for catheters.

The catheter 100 also includes a releasable wire-securing device 132 that is coupled to a portion of the second portion 112 of the longitudinal catheter body 102. The releasable wire-securing device 132 is for releasably securing at least one guide wire (e.g., first guide wire 118 or second guide wire 128) relative to the longitudinal catheter body 102. For example, the wire-securing device 132 may be used to hold the second guide wire 128 within the second rapid exchange lumen 116 during introduction and placement of the second guide wire 128 into the patient. The releasable wire-securing device 132 may be a wire clip. In one embodiment, the releasable wire-securing device 132 may be a plate 136 coupled to the catheter body 102 orthogonally to the axis of the longitudinal catheter body 102. As used herein, the term “coupled” includes coupling via a separate object and includes direct coupling. The term “coupled” also encompasses two or more components that are continuous with one another by virtue of each of the components being formed from the same piece of material. Also, the term “coupled” may include chemical, such as via a chemical bond, mechanical, thermal, or electrical coupling. The releasable wire-securing device 132 may have at least one deformable notch that is sized and configured to form an interference fit with a corresponding guide wire to be used therein. The wire securing device may be able to secure one or two wires. The releasable wire-securing device 132 of FIGS. 1-2 is shown with two notches 138. A Tougy-Burst attachment might also be used. Other distal guide wire-securing devices such as magnetic or other types of releasable mechanical fixation devices may also be coupled to the distal end of the catheter.

A first radiopaque marker 140 is attached to the longitudinal catheter body 102 proximate to the catheter tip 106. A second radiopaque marker 142 (FIG. 2) may be positioned proximate to the first port 124 of the second rapid exchange lumen 116. The radiopaque markers 140, 142 may be formed from any radiopaque material, e.g., gold, tungsten, silver, platinum, and alloys thereof, plastics, polymers, and other synthetic materials. The second radiopaque marker 142 may be constructed in such a manner as to indicate two dimensionally the plane of guide wire exit to facilitate vessel bifurcational access.

Referring now to FIG. 2, a catheter 100 is presented that is analogous in most respects to that of FIG. 1, except the first port 124 of the second rapid exchange lumen 116 is displaced more proximally compared to FIG. 1. In addition, the wire-securing device 132 is shown with the second guide wire 128 disposed within a notch 138. Also, the second radiopaque marker 142 has been added proximate to the first port 124 of the second rapid exchange lumen 116.

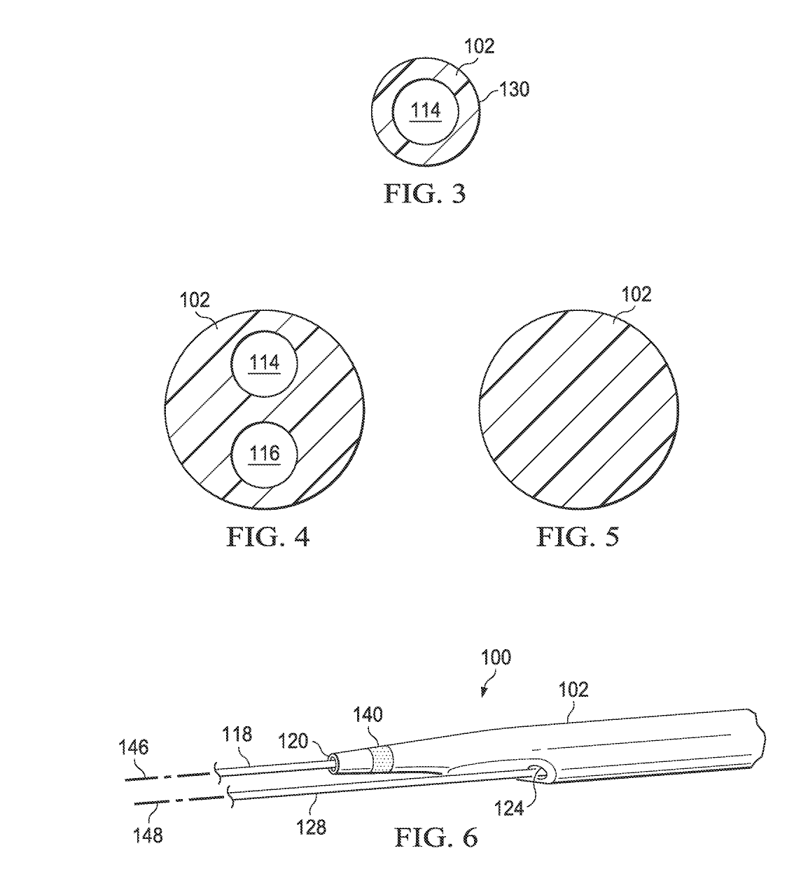

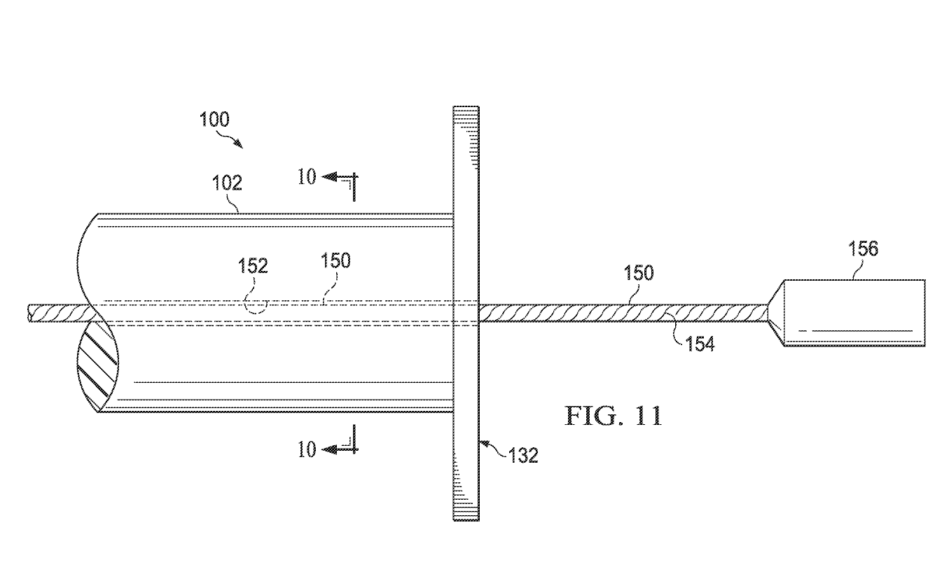

A number of cross sections taken along the length of the longitudinal catheter body 102 are shown in FIGS. 3, 4, and 5. As shown in FIG. 3, the longitudinal catheter body 102 has only the first rapid exchange lumen 114 near the catheter tip 106 distal to the first port 124. As shown in FIG. 4, the first portion 110 proximal to the first port 124 has both the first rapid exchange lumen 114 and the second rapid exchange lumen 116 formed in the longitudinal catheter body 102. The diameters of the first rapid exchange lumen 114 and the second rapid exchange lumen 116 may be the same or different sizes as desired for different applications. Moreover, as discussed in connection with FIGS. 8-11, stiffening members may be added. As shown in FIG. 5, the second portion 112 does not include the first rapid exchange lumen 114 and the second rapid exchange lumen 116 and is shown as a solid portion of the catheter body 102 but could be hollow in some designs.

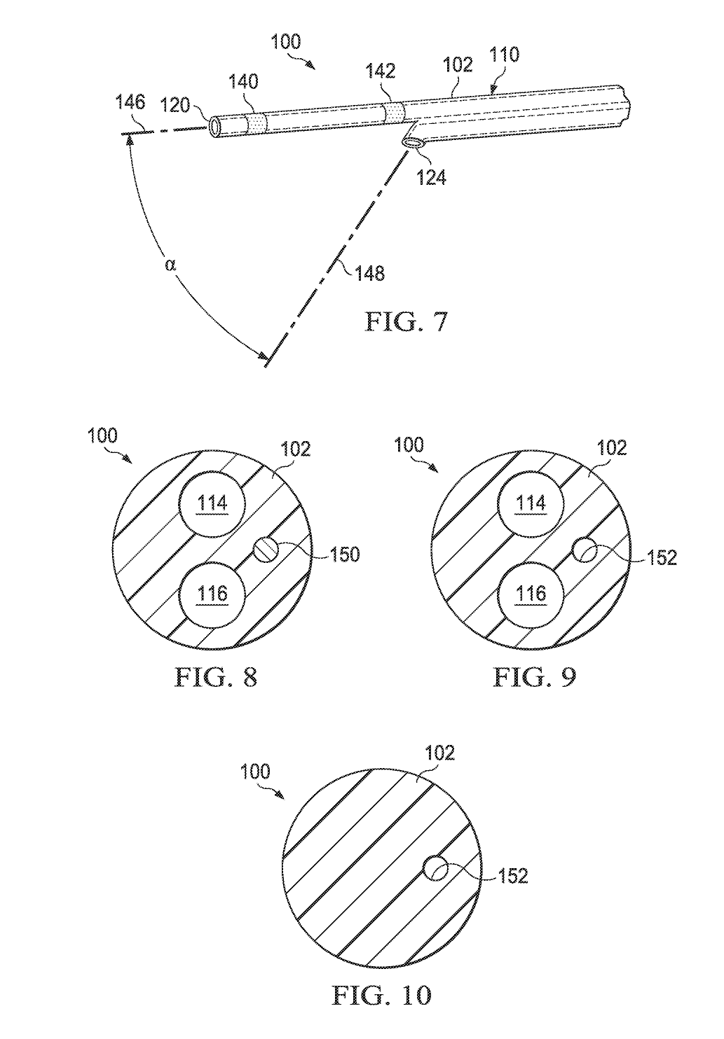

The catheter 100 may be used to place the second guide wire 128 parallel to the first guide wire 118 or may be used to position the second guide wire 128 at an angle. Referring now to FIG. 6, the first port 120 of the first rapid exchange lumen 114 has an exit axis 146 parallel to a longitudinal axis of the longitudinal catheter body 102 and the first port 124 of the second rapid exchange lumen 116 has an exit axis 148 that is also substantially parallel to the longitudinal axis of the longitudinal catheter body 102. In other embodiment, the exit axis of the first port 124 may be angled as discussed in connection with FIG. 7 below.

The guide wires 118, 128 may be any of numerous sizes as appropriate for the particular application involved. For example, a 0.014 inch guide wire may be used for coronary arteries. A stiffer wire (because of the nature of the material or increased size) may be used for the second guide wire 128 when it is difficult to pass a catheter past a restriction or blockage. The second guide wire 128 for use in the second rapid exchange lumen 116 may be the same size or a different size from the first guide wire 118 in the first rapid exchange lumen 114. For many applications, the guide wires 118, 128 will vary in diameter from 0.010 to 0.038 inches, but other dimensions are possible. The guide wires 118, 128 may be formed from any suitable material, such as stainless steel, nitinol, plastics, polymers, and suitable combinations thereof, etc.

In operation, according to one illustrative embodiment, the first guide wire 118 has already been introduced into the patient for a procedure or for one reason or another. A decision is made that a second guide wire 128 is desired. This may be ultimately to replace the first guide wire 118 or to have stiffer or more flexible wire or to gain access to another area at an angle (discussed further below in connection with FIG. 7). The catheter 100 is presented and the second guide wire 128 is back loaded through first port 124 or front loaded through port 126 such that the distal end of the second guide wire 128 is within the second rapid exchange lumen 116 and the proximal end of the second guide wire 128 exits the second port 126. A portion of the second guide wire 128 proximal to the distal end may be clipped or otherwise secured within wire-securing device 132. The proximal end of the first guide wire 118 is introduced into first port 120 of the first rapid exchange lumen and is advanced so that a proximal portion exits the second port 122. The catheter 100 is then advanced along the first guide wire 118 until the catheter is in a desired position.

The desired position may be determined using fluoroscopy to see when the first radiopaque marker 140 or second radiopaque marker 142 is in a desired location. Once the catheter is in the desired location, the second guide wire 128 is released from the releasable wire-securing device 132, and the operator advances the distal end of the second guide wire 128 to the final desired location. While holding the guide wires 118, 128 the catheter 100 is removed from the patient's body and both guide wires 118, 128 are in place. If the second guide wire 128 is a replacement, the first guide wire 118 may have been previously pulled into the first rapid exchange lumen 114 and removed with the catheter 100.

In another illustrative, non-limiting example, vascular access has been obtained, and a coronary guide catheter is advanced to the left main coronary artery. A first guide wire 118, e.g., 0.014 inch guide wire, is advanced across a severe stenosis in left anterior descending artery (LAD) that is distal to a large diagonal branch that also has a severe stenosis. The LAD stenosis is pre-dilated with a coronary balloon catheter inserted over the first guide wire 118. The balloon catheter is then exchanged for a coronary stent over the first guide wire 118. Unfortunately, in this illustrative scenario, the stent cannot be advanced across the stenosis and so it is removed. At this point, the operator wants a stiffer guide wire, but does not want to endure the additional time and effort to run a second guide wire by traditional means and wishes to avoid possible entanglement of the two guide wires. The operator thus selects the catheter 100.

The catheter 100 is loaded now with an extra stiff 0.014 inch guide wire, as the second guide wire 128, is back loaded through the first port 124 of the distal end of the second rapid exchange lumen 116 and pulled until the proximal end is out of the second port 126 and the distal end of guide wire 128 is proximal to port 124 with the distal end entirely within lumen 116 and the proximal end of the guide wire 128 is attached to the wire-securing device 132. The second guide wire 128 could also be front loaded through port 126 and advanced until the distal end exists port 124 then pulled back to lie within lumen 116 followed by securing of the proximal end of the guide wire 128 to the wire-securing device 132. The catheter 100 is advanced over the first guide wire 118 using the first rapid exchange lumen 114. Once the catheter 100 crosses the stenosis, the extra stiff wire (the second guide wire 128) is advanced to a distal position in the LAD. The catheter 100 is then removed leaving both guide wires 118, 128 in place. The operator now has an option to advance the coronary stent over the first guide wire 118 using the second guide wire 128 as a buddy wire to facilitate crossing the stenosis. Alternatively, the second guide wire 128 may be used with the first guide wire 118 as a buddy wire. Once the stent is in good position, the buddy wire is pulled proximal to the stent and the stent is deployed and the delivery device removed leaving both wires 118, 128 in place. The diagonal artery stenosis may be addressed with a modified version of the catheter 100 as described in connection with FIG. 7. It should be appreciated that the procedures discussed herein can be done by a single operator as contrasted with an over-the-wire design.

Referring now to FIG. 7, the first port 120 of the first rapid exchange lumen 114 has an exit axis 146 parallel to a longitudinal axis of the longitudinal catheter body 102 and the first port 124 of the second rapid exchange lumen 116 has an exit axis 148 that is angled relative to the longitudinal axis of the longitudinal catheter body 102 (and thus to the first exit axis 146). The angle, alpha (α) may be a few degrees all the way to 90 degrees or anything in between. For example, without limitation, the angle formed by the longitudinal axis of the first port 124 relative to the longitudinal axis of the longitudinal catheter body 102 may be 5, 10, 15, 20, 25, 30, 35, 40, 45, 50, 55, 60, 65, 70, 75, 80, 85, or 90 degrees or any number between these. While not shown flush or indented, it should be understood that the first port 124 of the second repaid exchange lumen 116 may be so formed.

Now continuing the previous illustrative operation, if the diagonal artery or bifurcation cannot be easily accessed with the first guide wire 118, which is more flexible, the catheter 100 (FIG. 1) can be used to deliver another type of guide wire, e.g., another 0.014 inch wire, to the LAD as discussed above and the new guide wire 128 used to steer to the diagonal artery. Alternatively, the catheter 100 shown in FIG. 7 with an angled exit for first port 124 of the second rapid exchange lumen 116 may be used. In this situation, the catheter 100 is inserted over the first guide wire 118 with the second guide wire 128 within the catheter 100 as before and advanced to the desired position. The catheter 100 is positioned with the second radiopaque marker 142 proximate to the diagonal artery. The second guide wire 128 is released from the wire-securing device 132 and advanced out of the catheter 100 at the angle a and is directed into the diagonal artery branch. The catheter 100 may then be removed and a stent delivered using the second guide wire 128.

According to another illustrative, non-limiting embodiment, the exchange of guide wires of different sizes is desired. For example, suppose a right common femoral artery vascular access is obtained using a 45 cm sheath. The sheath is advanced over an initial guide wire to the left common femoral artery. A 0.035 guide wire, first guide wire 118, is then advanced across an occluded left superficial femoral artery (SFA) and is treated with angioplasty and stenting. The operator would then like to treat blockages in the anterior and posterior tibial arteries but this interventional equipment will require a small guide wire. The catheter 100 is loaded with a second guide wire 128 that is smaller, e.g., 0.014 wire, and as before the catheter 100 is advanced. The second guide wire 128 is moved into the anterior or posterior tibial artery. The catheter is then removed. The 0.035 wire is left in the popliteal artery or pulled more proximal and intervention proceeds over the 0.014 wire, second guide wire 128. In a variation of this procedure, the angled exit catheter or first port 124 of the second rapid exchange lumen 116 in FIG. 7 could be used to advance the second guide wire 128 into an angulated take off of the anterior tibial artery. It should be noted that the use of catheter 100 also avoids the potential of inadvertently passing the second guide wire underneath the struts of a recently deployed stent.

While examples have been given involving coronary and peripheral arteries, it should be understood that the catheter 100 may be used in various parts of the body and is not limited to coronary and peripheral arteries. Other parts might include, without limitation, any and all accessible peripheral, visceral, renal, extra-cranial, intra-cranial arteries and veins, bile ducts, tracheo-bronchial tubes or any and all interventions that require initial placement of a guide wire.

In some embodiments, it may be desirable to stiffen the catheter 100 or stiffen portions of the catheter 100. Referring now to FIG. 8, another cross section is shown taken along 8-8 in FIG. 1. This embodiment is analogous to that presented in connection with FIG. 1 except that a stiffening member 150 has been added. The stiffening member 150 may run the whole length or only a portion of the length of the longitudinal catheter body 102. The stiffening member 150 may be a wire or any member of suitably stiff material and may be formed from any of one or more materials, e.g., stainless steel, nitinol in the super-elastic phase, or any other suitable material.

Referring now to FIGS. 9-11, another illustrative embodiment of the catheter 100 is presented that is analogous to the catheter of FIG. 1, except a stiffening-member lumen 152 has been formed in the longitudinal catheter body 102. FIG. 9 is taken at line 9-9 in FIG. 1. FIG. 10 shows a lateral cross section on the second portion 112 and shows that only the stiffening-member lumen 152 is in that portion. FIG. 10 alone shows the stiffening member 150 inserted into a portion of the stiffening-member lumen 152. The wire-securing device 132 has an aperture or channel that allows the wire stiffening member 150 to extend there through. The stiffening member 150 includes proximal treading 154 and a rotating cap 156 to advance or retract the stiffening member 150 relative to the stiffening-member lumen 152. Other devices may be used to control the movement of the stiffening member 150. In this way, the depth of penetration of the stiffening member 150 into the stiffening-member lumen 152 may be controlled and along with it the point at which the catheter becomes stiffer.

According to an illustrative embodiment, a catheter could also be used to introduce two guide wires by loading both guide wires into the catheter and securing into position. The catheter is then advanced to the distal end of the guide catheter or guide sheath. The first guide wire is released and positioned in place. The second guide wire is then released and advanced into a bifurcation vessel such as an LAD/diagonal bifurcation. An advantage of this approach, which is also an advantage of the catheter when adding a second guide wire to a guide wire already in place, is that one avoids the guide wires from becoming entangled since the guide wires are kept separated by the catheter. Once the guide wires become entangled one would be unable to advance interventional equipment over either guide wire and if one is unable to get them untangled one has to pull both guide wires and start over. Also once a stent is in place one does not want to lose the guide wire position across the true stent lumen until one is totally finished with the intervention. If one needs to pass a second wire, the catheter of this embodiment not only ensures that the second wire will not pass underneath the stent struts but also keeps the guide wires from entanglement as noted above. This is in addition to not having to duplicate all the work of placing the first wire.

Although the present invention and its advantages have been disclosed in the context of certain illustrative, non-limiting embodiments, it should be understood that various changes, substitutions, permutations, and alterations can be made without departing from the scope of the invention as defined by the claims. It will be appreciated that any feature that is described in a connection to any one embodiment may also be applicable to any other embodiment.

1. A catheter for introducing a second guide wire into a patient, the catheter comprising:
a longitudinal catheter body having a distal end with a catheter tip and a proximal end, wherein the longitudinal catheter body has a first portion and a second portion that is proximal to the first portion;
a first rapid exchange lumen formed in the first portion of the longitudinal catheter body, the first rapid exchange lumen having a first port proximate the catheter tip and a second port on a medial portion of the longitudinal catheter body;
a second rapid exchange lumen formed in the first portion of the longitudinal catheter body, the second rapid exchange lumen having a first port proximate the distal end of the longitudinal catheter body that is proximal to the first port of the first rapid exchange lumen, and wherein the second rapid exchange lumen has a second port on a medial portion of the longitudinal catheter body; and
a releaseable wire-securing device coupled to a portion of the second portion of the longitudinal catheter body for releasably securing at least one guide wire relative to the longitudinal catheter body.

2. The catheter of claim 1, wherein the first port of the second rapid exchange lumen is displaced proximally from the first port of the first rapid exchange lumen by at least one percent of a length of the longitudinal catheter body.

3. The catheter of claim 1, wherein the first port of the second rapid exchange lumen is displaced proximally from the first port of the first raid exchange lumen by at least five percent of a length of the longitudinal catheter body.

4. The catheter of claim 1, wherein the first port of the second rapid exchange lumen is displaced proximally from the first port of the first raid exchange lumen by at least five percent of a length of the longitudinal catheter body and less than 50 percent of the length of the longitudinal catheter body.

5. The catheter of claim 1, wherein the first port of the first rapid exchange lumen has an exit axis parallel to a longitudinal axis of the longitudinal catheter body and the first port of the second rapid exchange lumen has an exit axis that is angled relative to the longitudinal axis of the longitudinal catheter body.

6. The catheter of claim 1, wherein the first port of the first rapid exchange lumen has an exit axis parallel to a longitudinal axis of the longitudinal catheter body and the first port of the second rapid exchange lumen has an exit axis that is angled relative to the longitudinal axis of the longitudinal catheter body by at least ten degrees.

7. The catheter of claim 1, wherein the first port of the first rapid exchange lumen has an exit axis parallel to a longitudinal axis of the longitudinal catheter body and the first port of the second rapid exchange lumen has an exit axis that is angled relative to the longitudinal axis of the longitudinal catheter body between 10 and 90 degrees.

8. The catheter of claim 1, wherein the first port of the second rapid exchange lumen has an angled entry way.

9. The catheter of claim 1, wherein the releasable wire-securing device comprises a wire clip.

10. The catheter of claim 1, wherein a first inside diameter of the first rapid exchange lumen equals a second inside diameter of the second rapid exchange lumen.

11. The catheter of claim 1, further comprising a stiffening member disposed within the longitudinal catheter body for increasing stiffness of the catheter along at least a portion of the catheter.

12. The catheter of claim 1, wherein the longitudinal catheter body is formed with a stiffening-member lumen along at least a portion of the longitudinal catheter body, and further comprising a stiffening member disposed within the stiffening-member lumen.

13. The catheter of claim 1, further comprising a first radiopaque marker proximate the catheter tip and a second radiopaque marker proximate the first port of the second rapid exchange lumen.

14. A catheter for introducing a second guide wire into a patient, the catheter comprising:
a longitudinal catheter body;
a first rapid exchange lumen formed in the longitudinal body and having a first port and a second port that is proximal to the first port;
a second rapid exchange lumen formed in the longitudinal body and having a first port and a second port that is proximal to the first port of the second rapid exchange lumen;
a releasable wire-securing device associated with a portion of the longitudinal catheter body for releasably holding one or more wires exiting the second port of the first repaid exchange lumen or the second rapid exchange lumen.

15. The catheter of claim 14, wherein the first port of the second rapid exchange lumen has an exit axis that is angled relative to a longitudinal axis of the longitudinal catheter body.

16-20. (canceled)
